(19) United States
(12) Patent Application Publication
(10) Pub. No.: US 2026/0294631 A1
(43) Pub. Date: Oct. 1, 2026

(54) MEDICAL IMPLANT WITH COMPRESSIBLE JOINT

(71) Applicant: WALDEMAR LINK GMBH & CO. KG, Hamburg (DE)

(72) Inventors: Helmut D. LINK, Hamburg (DE); Eckhard BAUER, Kiel (DE)

(73) Assignee: WALDEMAR LINK GMBH & CO. KG, Hamburg (DE)

(21) Appl. No.: 19/478,267

(22) PCT Filed: Mar. 22, 2024
(86) PCT No.: PCT/EP2024/057748

§ 371 Date: Oct. 24, 2025

Related U.S. Application Data
(60) Provisional application No. 63/500,955, filed on May 9, 2023.

(51) Int. Cl. A61F 2/30; A61F 2/32; A61F 2/38

(52) U.S. Cl. CPC A61F 2/30734; A61F 2/30771; A61F 2/3094; A61F 2/32; A61F 2/38; A61F 2002/30176; A61F 2002/30326; A61F 2002/30331; A61F 2002/30505; A61F 2002/30537; A61F 2002/30736; A61F 2002/30784; A61F 2002/3093; A61F 2310/00017; A61F 2310/00023; A61F 2310/00029; A61F 2310/00089; A61F 2310/00131

(57) ABSTRACT

Medical implant, preferably for a joint endoprosthesis, being sleeve-shaped comprising a wall (2) surrounding a channel extending through the sleeve (10). The wall (2) is comprised of a plurality of wall segments (20) arranged in a circumferential direction. Neighboring wall segments (20) are connected at their adjacent edges (21, 22) facing each other by a compressible joint (3) which are configured for reducing a separation distance between said neighboring wall segments (20) of the wall (2). They comprise a leaf spring (30) spanning an interspace between said neighboring wall segments (20), thereby form an elastic spring connection. Thereby, the width of the separating interspace can be reduced in an elastic manner determined by the leaf spring. Consequently, circumference of the sleeve is reduced. As a result, the overall diameter (width) is reduced such that the whole sleeve is elastically compressed and will be enabled to fit into a smaller cavity.

### FIELD OF INVENTION

The invention relates to a medical implant, in particular for joint endoprosthesis, specifically a sleeve shaped implant comprising a wall surrounding a channel extending through the sleeve. More particularly, the sleeve may be configured as an implant of the augment type, and a set of medical implants having different sizes may be provided.

### BACKGROUND

Due to diseases, injuries and/or wear, in particular due to high age, patients often require endoprosthesis to restore joints such as knee, hip, shoulder etc. In many cases additional medical implants are required to restore defective bone material and to provide a sufficiently strong support, in particular but not limited to for anchoring the endoprosthesis in the bone. For achieving a proper fitment and a reduction of risk to damage the bone during insertion, said medical implants can be compressible for better adaption to the size of the bone defect.

It is known to provide medical implants in the form of a cone-shaped augment device that is provided with a bending joint incorporated in the wall of the cone (US 2019/0070008 A1). Due to a force applied on the circumference of the wall a compression will be effected and subsequently the width of the cone will be reduced for fitting into a cavity of the bone. However, achieving a sufficient compression often requires a force that is larger than the remaining bone can accommodate. This is of particular concern with bones with substantial defects as they have even less healthy bone material that can be relied on to withstand the force. In other words, these bones are substantially weakened and are compromised in terms of being enabled to withstand the force of the compressed implant. This creates a risk of further damaging the remaining parts of the already damaged bone and leading subsequently to serious complications for the patient.

Thus, there is a need for an improved medical implant that provides greater compression for better fitment with lower compression force. It is thus an object of the invention to provide such a medical implant.

### SUMMARY

The solution according to the invention resides in the features of the independent claims. Advantageous embodiments are the subject matter of the dependent claims.

A medical implant, in particular for a joint endoprosthesis, being sleeve-shaped comprising a wall surrounding a channel extending through the sleeve from a bottom to a top of the sleeve, is provided according to the invention wherein the wall is comprised of a plurality of wall segments arranged in a circumferential direction, wherein neighboring wall segments are connected at their adjacent edges by a compressible joint, the compressible joint being configured for reducing a separation distance between said neighboring wall segments of the wall and comprising a leaf spring spanning an interspace between the respective neighboring wall segments such as to form an elastic spring connection between the neighboring wall segments. This leads to a reduction of circumference of the wall and thus effecting a compression of the channel.

The core aspect of the invention is having the wall of the sleeve being segmented into wall segments with a separating interspace between neighboring wall segments in combination with the leaf spring spanning the interspace between the adjacent edges of neighboring wall segments. The circumference of the sleeve is thus defined by the sum of the circumferential length of the wall segments plus the separation distance of the interspace. The leaf spring forms an elastic spring connection between the neighboring wall segments, thus allowing-upon application of a compressing force on the circumference of the sleeve-to bring neighboring wall segments closer to each other, against an opposing force of the leaf spring. In other words, the leaf spring is configured to provide opposing force against reduction of the separation distance. Thereby, the width of the separating interspace can be reduced in an elastic manner determined by the leaf spring, and consequently the circumference of the sleeve is reduced. As a result, the overall diameter (width) of the sleeve is reduced such that the whole sleeve is elastically compressed and will be enabled to fit into a smaller cavity.

In a practical example, at typical dimensions for the medical implant being a tibial cone, such a compressible joint with one leaf spring will allow for a reduction of about 3 mm for the circumference, which will add up to 12 mm (or 18 mm) if the wall is divided into four (or six wall) segments with a corresponding number of compressible joints. Accordingly, a rather high reduction of the circumference and consequentially width/diameter of the medical implant can be achieved, allowing an adaption to even rather small cavities in smaller bones.

The force required for achieving this compression effect of the compressible joint (which may also be termed as compression joint) is determined by the properties of the leaf spring and its attachment to the adjacent edges of the neighboring wall segments, in particular by effective length and width of the leaf spring as well as by the elastic properties of the material used therefore. Thus, the properties of the compressible joint are mainly decoupled from the properties of the wall segment. This allows for a greater degree of freedom concerning adapting and choosing parameters of the compressible joint as it was possible with compressible cone implants according to the prior art. The independence gained thereby can be used e.g., for a more liberal selection of materials to be used for the wall segments and/or the leaf spring.-But even if the material of the leaf spring and/or its thickness are defined by that of the wall segments, still compressibility can be selected over a rather large range by adjusting the relevant dimensioning, particularly effective length and/or width, of the leaf spring.

Another important advantage of this independence is that it is now possible to provide decouple sizing of the implant from its elasticity. This allows, e.g., smaller implants to have compressible joints which are more elastic. This is particularly an advantage with respect to application of the medical implant at bones having substantial defects and therefore, due to less healthy bone mass, are compromised in terms of force bearing capability. Conventionally, where the wall itself is used as bending joint for achieving compressibility as known in the prior art, providing a smaller implant often leads to an increase of the required compression force (since smaller components are less springy than larger components of the same general shape and material), and such unwanted increase of the compression force may be a source of risk for the bone and the health of the patient. This major drawback of the known medical implants can be overcome by the present invention.

Since the parameters relevant for the compressible joint are those of the element forming the leaf spring and its flexure bearings, not of the wall segments proper, adjustments of properties of the joint (e.g., its stiffness) can be made rather independently of wall (segment) properties. But even if thickness and material are determined by that of the wall segments, still compressibility can be selected in particular by adjusting length and/or width of the leaf spring, without adverse influence on the constitution of the wall segments.

Preferably, the leaf spring is connected to at least one of the respective neighboring wall segments, preferably by a flexure bearing. Thereby, a double effect is achieved: the leaf spring is solidly affixed to the respective of the wall segment, and further, owing to this fixation, the leaf spring acts like a cantilever beam. This allows for a well-determined elastic behavior of the leaf spring upon application of a compressional force. As a result, the flexure bearing formed thereby does also contribute to the elasticity in addition to the leaf spring. Further, the flexure bearing ensures a smooth transition. Moreover, such fixation also ensures that the leaf spring does not decouple from the respective edge, e.g., if the sleeve happens to be expanded by the surgeon. By virtue of this, the medical implant becomes more robust in terms of handling prior to implantation. Preferably, the flexure bearing is attached at at least one end of the leaf spring. Further preferably, the leaf spring is at both ends connected to the respective wall segment. This enhances the aforementioned effects and avoids any drawbacks that may be arise due to asymmetrical fixation of the leaf spring.

Advantageously the leaf spring is positioned within the interspace between the respective adjacent wall edges. Thereby the benefit can be realized that no protruding parts are required for the compressible joint, thus minimizing irritation of surrounding tissue and bone material.

Advantageously, at either side of the leaf spring free space is provided forming compression slits. The compression slits provide thus free space in order to allow the leaf spring, e.g., formed as a crossbar lever, to be moved into closer proximity of the neighboring wall segments, namely closer to the adjacent edge of the respective wall segment. Thereby compression can be achieved without being blocked by the leaf spring abutting against the wall segment. The compression slits are preferably on one side open, e.g., one is open toward the top of the implant and the one on the other side of the leaf spring is open toward the bottom of the implant. Thereby a kind of interdigitated slits are formed allowing for an increase of the travel of the compressible joint. Further preferably, the compression slits on both sides of the leaf spring are preferably oriented such as to be in opposition to each other. Preferably, the compression slits are V-shaped. For a given (average) width of the compression slits, such V-shaping allows for an increase of the travel that can be accomplished by the compressible joint.

It is preferred that the compression slits are covered by tongues provided at the adjacent edges and/or the crossbar. Without such tongues, there could be a direct line-of-sight conduit be formed from the interior of the sleeve to the exterior via the compression slits, and bone cement that is to be applied in the interior of the sleeve for fixation of an endoprosthesis, in particular the stem thereof, may flow out and reach the exterior portion of the sleeve which is generally unwanted due the risk that outflowing bone cement would clog the pores of an external porous structure and thus prevent the bone from growing into said porous structure. By providing said tongues as a cover, such a direct conduit is no longer present, and an unrestricted outflow of bone cement is avoided. The tongues are advantageously configured such as to form a sealing gap. By virtue of such a sealing gap, outflow of bone cement from the interior to the exterior can be effectively avoided. To this end, the sealing gap preferably has a width of 0.5 mm or less in an uncompressed state of the medical implant, further preferably 0.4 mm or less. This provides a sufficient sealing effect against outflow of bone cement.

Preferably the leaf spring is formed as a unitary piece with at least one of the adjacent edges of the neighboring wall segments. This allows for a robust fixation of the leaf spring and an efficient manufacturing. Further, if the leaf spring is a unitary piece with both of the adjacent edges, then physical continuity between the two neighboring wall segments can be achieved via the leaf spring. This allows for the parts being contiguous so that they cannot become de-coupled or get lost, which facilitates manufacturing and handling of the medical implant and is further beneficial for long-term integrity of the medical implant. Owing to the independence achieved by the compressible link being configured with said leaf spring, even if due to the unitary piece characteristic thickness as well as material of the leaf spring are fixed by that of the wall segments, still compressibility can be widely selected by adjusting the dimensions, in particular length and/or width, of the leaf spring and its flexure bearings. This is a unique advantage in order to withstand higher loads and to avoid any risk of loosening or parts getting lost. Moreover, in a further preferable embodiment the medical implant, including its wall segments and leaf spring(s), is formed as a unitary piece. Thereby the complete medical device can be one piece which facilitates manufacturing as well as handling. Specific connecting elements are not required which is beneficial for a smooth surface without protuberances, thereby keeping low irritation of surrounding bone and tissue material. A further advantage is that no discrete parts can get missing, and no wrong or wrongly parts could be combined, thereby further enhancing safety for the patient.

In a preferred embodiment, the leaf spring may be configured as a crossbar, e.g., as a single crossbar. This has the advantage of a simple yet effective configuration. The crossbar is preferably arranged at an oblique (not parallel to the edge of the adjacent wall segment but in-plane) orientation. Thereby it can form a Z-shaped configuration with the adjacent edges of the neighboring wall segments, and therefore forms a simple and effective leaf spring. Further, an oblique arrangement is beneficial for providing a rather long and springy crossbar in a confined space. Preferably, the crossbar forms an acute angle with the adjacent edges of the neighboring wall segments, preferably being 10 degrees or less, further preferably 6 degrees or less, yet further 2.5 degrees or less, and being preferably at least 0.5 degrees, further preferably at least 1.5 degrees.

It is generally advantageous to have a rather large effective length of the leaf spring for achieving a more elastic compressible joint. Preferably, in the case of the leaf spring being configured as a crossbar it is dimensioned such to have its length corresponding to the local height of the medical implant, wherein the local height of the medical implant is defined by the distance between bottom and top of the wall at the respective location. In particular, a length is corresponding to the local height if it is similar in length, which usually means length differs from the local height by no more than 35 percent, preferably no more than 25 percent, further preferably no more than 20 percent.

However, the crossbar may also be configured to be folded and/or as multiple crossbars. Such a configuration allows for an increased effective length of the crossbar within the same interspace between the wall segments. An example of a folded crossbar is a double configuration having a V-shaped configuration. In this case, the crossbar is folded and attached with its ends to the respective adjacent edge of the wall segments. A multiple configuration further increases the effective length, e.g., to be trifold or more. It is important to note that in many instances the crossbar may be straight for the sake of simplicity and ease of manufacturing, however it could also be curved, bent or folded, as stated, in particular for achieving an increased effective length.

The effective length of the crossbar is the dimension of the crossbar along its main axis of extension which contributes to the elasticity. Typically, said main axis is the middle axis along the elongation of the crossbar. Said main axis follows the shape of the crossbar, i.e., if the crossbar is folded then the main axis will be folded, too.

For example, if two crossbars are provided, then preferably each is connected with one of their ends to the adjacent edge of the respective neighboring wall segment and is connected with their other (free) end to the other respective crossbar. An example for this is a V-like configuration. For multiple crossbars, the middle (third and if applicable any further) crossbar is connected with either end to the respective neighboring crossbar. Thereby, the spring leaf effect of this crossbar assembly is increased, and a larger travel for the compressible joint can be realized. By such a configuration the medical implant compressibility can be deliberately increased at a targeted location of such a double or multiple configurations. This is advantageous if the compressible joint shall be made considerably softer, requiring less compression force for the same travel, at least at one specific location.

Advantageously, a plurality of said compressible joints are provided. A higher number of the compressible joints allows for a corresponding larger amount of distance by which the circumference of the medical implant may be reduced. Further, the higher number allows for the medical implant to maintain the same general shape even in a compressed state. It is particularly beneficial to have at least four compressible joints so that at least one joint is located in either of the cardinal directions, being anterior, posterior, medial, and lateral (those terms are anatomical terms which are in common use also with respect to medical implants).

As it is known to a person skilled in the art, the terms “anterior” and “posterior” refer to the direction of the augment device into which it is designed to be implanted at a human body. “Anterior” relates to a forward-facing direction, and “posterior” to a rearward facing direction. Similarly, the term “lateral” refers to a direction substantially perpendicular to the anterior/posterior direction, namely to the direction away from a center of the body in a lateral direction, and “medial” refers to the opposite direction toward the center.

Advantageously, a plurality of the compressible joints are provided spaced equidistantly and/or equiangularly along the circumference of the wall. This allows for a more even distribution of the resulting reduction of the circumference and is further beneficial for maintaining the general shape of the implant if heavily compressed. If the implant has symmetry (in particular with respect to a middle plane, e.g., like an anterior-posterior and/or medial-lateral plane), then the compressible joints preferably are located symmetrically, further preferably mirror-symmetrically with respect to the middle plane.

In a preferred embodiment, the wall has an inner face to the channel and an outer face on the opposite side of the wall facing to the exterior, wherein the outer face is at least partially comprised of porous material configured for bone ingrowth. By virtue of the porous material, ingrowth of bony material is being promoted, thereby achieving an improved long-term stability. The inner face, on the other hand, is preferably solid. The solid face acts as a kind of bulkhead avoiding that any cement flows through the wall. This is particularly important due to bone cement that is to be applied in the interior of the sleeve in order to connect the wall to a stem of the endoprosthesis. Without such a bulkhead there would be the risk of the bone cement flowing from the interior through the wall and filling the porous material, thereby blocking ingrowth of bony material which would lead to the unwanted consequence of an unsatisfactory fixation of the medical implant to the surrounding bone.

Advantageously, the wall comprises pockets in which porous material is affixed. Thereby well-defined zones can be formed wherein the porous structure shall be present and where ingrowth of bone material shall happen. In most cases, the porous portion is formed as a unitary piece with the wall segments, which is a much-preferred embodiment. Further preferably, the porous structure is configured to be osteoconductive, and further optionally it may be provided with an osteoconductive coating. The remaining portions of the wall may be solid and therefore mechanically more robust and capable of bearing higher loads. Preferably, the pockets have such a depth that the porous structure has at least one, preferably two or more layers of pores for proper ingrowth of the bony material. The pores are preferably interconnected which allows for a stronger fixation by ingrown bony material.

The medical implant is preferably made of biocompatible material, preferably metal or zirconium, further preferably a material selected from a group comprising pure titanium like e.g., titanium grade 2 or titanium grade 4, a titanium alloy like e.g., Ti6Al4V or titanium grade 5(Ti6Al4V ELI) or grade 23, tantalum, cobalt chromium and stainless steel. These are biocompatible materials which provide sufficient strength and long-term stability for the medical implant.

In a particularly advantageous embodiment that may deserve independent protection, the medical device is formed by additive manufacturing, preferably by 3D printing. Thereby even complex shaped embodiments with a plurality of leaf springs, even complex shaped leaf springs, can be efficiently manufactured including any porous structure. As a result, reliable and efficient manufacturing can be performed in an economical manner.

Preferably the medical implant is configured as an augment device for an endoprosthesis, wherein the channel is configured for receiving a stem of said endoprosthesis, preferably an articulated joint endoprosthesis. For proper anchoring of the endoprosthesis in the adjacent bone, typically a long bone, a proper seating at the bone is necessary. If the bone is defective, either due to illness or due to removal of fractured or otherwise defective bone material, then an augment is beneficial for providing proper support for the endoprosthesis including its stem. The medical implant as an augment device according to the present invention is particularly suited for usage at sites having a rather high degree of defective bone material since a rather large compression of the medical implant for better fitment can be achieved with a comparatively low force due to the unique design of the compressible joints with their leaf springs.

Particularly, the medical implant according to the present invention may be used as an implant cone for an articulated joint endoprosthesis, preferably as a tibial, femoral, or humeral implant cone, or as a dental cone.

The invention further relates to a set of medical implants as described herein, the set comprising a plurality of such implants in different sizes comprising at least a large medical implant and a small medical implant, the large medical implant having a larger channel width than the small medical implant, thereby allowing fitment of a stronger and bigger endoprosthesis having a corresponding bigger stem for proper anchoring. By virtue of such a set, a variety of different bone sizes can be provided with a properly dimensioned medical implant. Preferably, a larger medical implant also features a greater height than a small medical implant.

By different sizes it is understood that the medical implant differs in diameter/width and/or height and/or the dimension of the free space in the interior forming the channel.

By virtue of such a set having at least one larger and one smaller implant, a variety of different bone sizes and defects can be provided with a properly dimensioned medical implant. Preferably, within said set the smaller medical implant features a leaf spring having a higher length/width ratio than that of the leaf spring of the larger medical implant. Such a higher length/width ratio could be realized by the leaf spring having a greater length or a smaller width, or both. Thereby it can be achieved that a medical implant of the smaller size can have the same elasticity compared to that of the larger medical implant, which is often desirable (or even a higher elasticity if so wished).

Further, the invention relates to a method for treating a subject in need of bone repair, the method comprising implanting into the subject the medical implant as described in the fore-going.

### BRIEF DESCRIPTION OF THE DRAWINGS

The invention is explained in more detail by way of examples in conjunction with the accompanying drawing showing advantageous embodiments. In the drawing:

FIG. 1 is a frontal view of a first exemplary embodiment of the medical implant;

FIG. 2 is a cross-section at a top portion along the line II-II of FIG. 1 showing a plurality of compressible joints;

FIG. 3 is a cross-section at a middle portion along the line III-III of FIG. 1 showing a plurality of compressible joints;

FIG. 4 is a schematic view showing a medical implant according to the invention in situ;

FIG. 5 is an enlarged detail of FIG. 2 as indicated by the dashed line marked by “V” in FIG. 1;

FIG. 6 is a surface development of a portion of a wall of the medical implant;

FIGS. 7A, B show details of the compression joints in a non-compressed and compressed state;

FIGS. 8A-H show views of various examples for a leaf spring between two adjacent wall segments;

FIGS. 9A-H show perspective views of the examples of FIGS. 8A-H being provided with additional tongues;

FIG. 10 shows a frontal view of a second exemplary embodiment of the medical implant;

FIGS. 11A, B show a schematical perspective bottom view of the second exemplary embodiment having its compressible joints in a non-compressed and compressed state;

FIG. 12 is a lateral side view of the second exemplary embodiment having a compressible joint having a double crossbar as a leaf spring;

FIG. 13 is a partial side view from lateral of the second exemplary embodiment having said compressible joint with a double crossbar as a leaf spring;

FIG. 14 shows a set comprising medical implants of the second exemplary embodiment in different sizes;

FIG. 15 shows a top view of the set of FIG. 14; and

FIG. 16 shows length and width dimensions of the leaf spring for the differently sized medical implants of the set.

### DETAILED DESCRIPTION

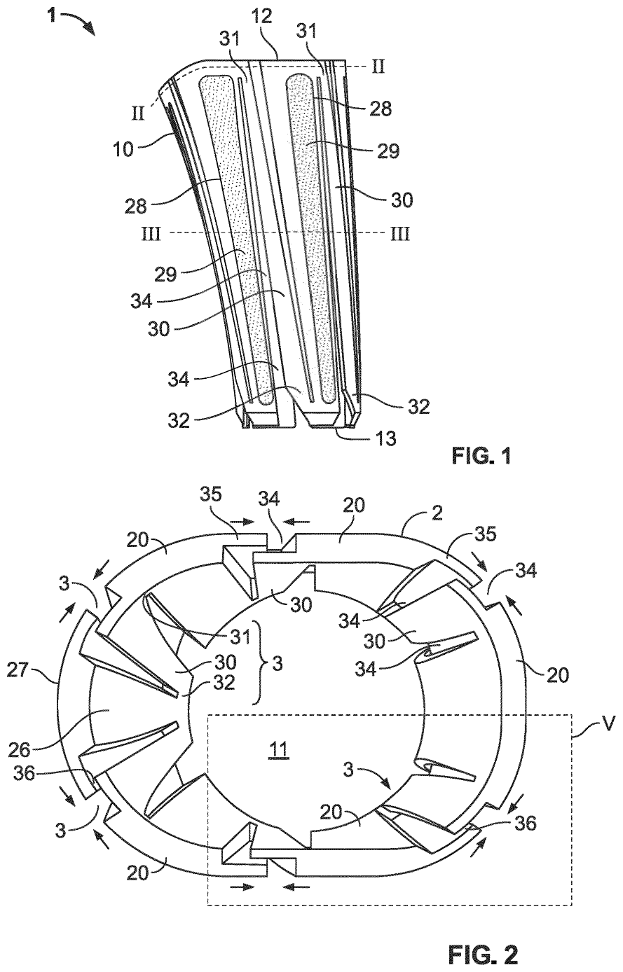

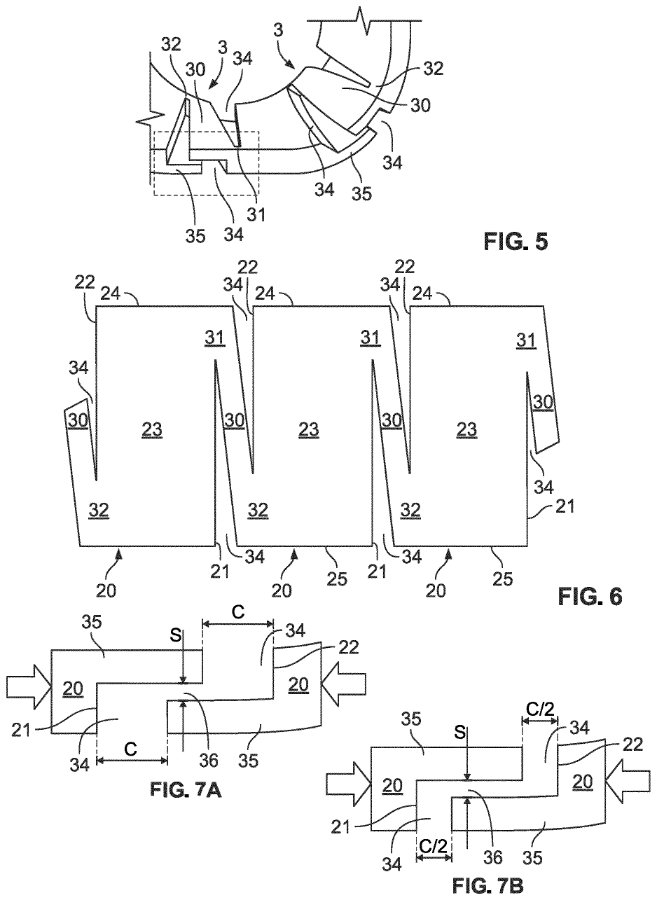

A first embodiment of a medical implant according to the invention is shown in FIG. 1 to 7. The medical implant of this first embodiment is preferably an augment device for a proximal portion of a tibia bone. It is manufactured from biocompatible metallic material, for example titanium grade 5. It may be manufactured by employing a 3D-printing technique. Machines for such a 3D-printing of metallic material are commercially available, e.g., Arcam EBM machines from the manufacturer GE Additive.

The augment device 1 according to the first embodiment is preferably of a generally conic form and is configured as a sleeve 10 having a wall 2 surrounding a channel 11 which runs through the sleeve 10 from the top 12 of the sleeve 10 all the way to a bottom 13 of the sleeve 10. The augment device 1 is configured such as to be conically sized and shaped to fill a cavity in a bone, typically at or near an end portion of the bone. The direction indicators “top” and “bottom” relate to the medical implant, in the present case augment device, being oriented such that-in consideration of the generally conical form-the wider end is the top and the narrower end is the bottom. Which end will be the proximal end which will be the distal end depends on the implantation site, in particular whether the medical implant is to be received at a proximal or distal end of the bone concerned.

The medical implant according to the first exemplary embodiment is to be implanted into a proximal portion of the tibia 99 in the exemplary embodiment as depicted in FIG. 4. The augment device is placed into a cavity of an upper portion of the tibia 99, thereby strengthening the surrounding bone in order to enable a stable fit of the prosthesis. The tibial component 92 comprises a stem 94 configured to be anchored in a medullary channel of the tibia bone 99. The stem 94 extends through the channel 11 of the augment device 1.-Said knee endoprosthesis 9 further comprises a femoral component 90 configured for rotatable interaction with the tibial portion 92. Said femoral component 90 comprises a stem 91 to be placed into the distal end of a femoral bone 98. A similar augment device (not shown) like the depicted augment device can be provided at said distal end of the femoral bone 98, of course in an upside-down orientation wherein the wider “top” end of said augment device is to be placed at the distal end of the femoral bone 98.

The stem 94 (or 91) will be fixated in the channel 11 by means of bone cement (not shown). For this reason, an inner face 26 of the wall 2 forming the sleeve 10 is made to have a solid surface. Conversely, an outer face 27 of the wall 2 forming the sleeve 10 is to be fixated to the surrounding bone in a cementless manner. To this end, pockets 28 are provided at the outer face 27 of the wall 2, said pockets 28 being filled with porous material 29. Said porous material 29 is configured to promote ingrowth of bony tissue. To this end, the porous material is configured to provide at least one, preferably multiple layers of pores, with pores ranging between 0.3 and 1.5 mm in width, preferably between 0.5 and 1 mm. Thereby, a stable long term fixation of the medical implant into the bone can be achieved.

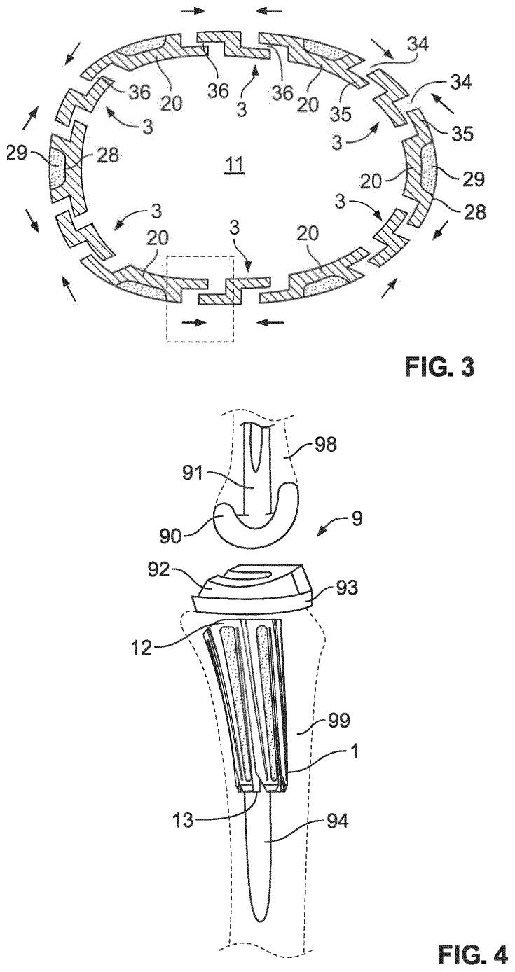

As clearly shown in the sectional views of FIG. 2 and FIG. 3, the wall 2 is comprised of a plurality of wall segments 20, said wall segments 20 forming the wall 2 that circumscribes the channel 11. Each wall segment 20 has a plate-like main section which may be essentially planar or curved and features a top edge, bottom edge and two side edges 21, 22.

Reference is now made to FIG. 6 which shows a surface development of a portion the wall 2 along its circumference in a partly compressed state. Said surface development shows three of the wall segments 20 which in total are approximately equivalent to a portion corresponding to a half of the circumference of the wall 2. Each wall segment 20 comprises a main section 23 (planar in the surface development as shown in FIG. 6, although in reality it may be planar or curved to follow the circumference as indicated in FIGS. 2 and 3) having a top edge 24 and a bottom edge 25 as well as two side edges 21, 22. Due to the wall segments 20 being arranged in series next to each other, two neighboring wall segments 20 have their side edges 21, 22 being adjacent to each other. The adjacent side edges 21, 22 of neighboring wall segments 20 are distanced from each other by an interspace.

A compressible joint 3 joins said side edges 21, 22 of neighboring wall segments 20. It comprises an example for the leaf spring 30 being configured as a simple crossbar 37 which is arranged diagonally in said interspace between the two adjacent side edges 21, 22 of the neighboring wall segments 20 and is positioned obliquely to connect an upper portion of the side edges 21 at one of the neighboring wall segments 20 to a lower portion of the side edge 22 on the other neighboring wall segment 20. Either end of the leaf spring 30 is attached to the respective one of the neighboring wall segments 20 by a flexure bearing 31, 32 located at a top or bottom portion of the respective side edge 21, 22, respectively. Between the leaf spring 30 and the respective adjacent side edge 21, 22 an acute angle is formed, approximately about 2 degrees in the exemplary embodiment (exaggerated in the figures for clarification). Thereby, an elastic compressible joint 3 is formed having a generally Z-like appearance wherein the leaf spring 30 is formed as a crossbar 37. The flexure bearing 31, 32 and the leaf spring 30, here crossbar 37, may be formed as unitary piece, which in turn is preferably formed as unitary piece with the respective neighboring wall segment 20.

Owing to said compressible joint 3, under a compression force said two neighboring wall segments 20 can be moved toward each other by bending the leaf spring 30 and the respective flexure bearings 31, 32 in an elastic manner. For a typical medical implant of the first exemplary embodiment, each compressible joint can provide a travel of about 2 mm, which achieves for a plurality of six compressible joints (see the embodiment as depicted in FIG. 2) a total compression of six times 2 mm which is 12 mm. This is a considerable reduction of the circumference of the medical implant (and consequently reduction of its width) that facilitates placing of the medical implant into a tight bone cavity, e.g., of the tibia bone 99. The compression achieved at each of the plurality of compressible joints 3 of the medical implant 1 is visualized by arrows in FIGS. 2 and 3.

As it can be seen in particular in FIGS. 1, and 2, between the leaf spring 30, here crossbar 37, and the respective side edges 21, 22 a V-shaped compression slit 34 is formed at either side of the crossbar 37. It provides for the necessary clearance space for allowing the leaf spring 30 with its crossbar 37 to be bended under compression force such that the neighboring wall segments 20 can move with respective edges 21, 22 closer to each other, leading to a reduction of the effective width of the compression slits 34; a partly compressed state is shown in FIG. 6. A broader compression slit 34 thus allows a greater range of travel for the compressible joint.

However, wide compression slits 34 may lead to a formation of gaps in the wall 2, thereby forming unwanted conduits between the channel 11 in the interior of the sleeve 10 and the exterior of the sleeve. Such conduits through the compression slits 34 would carry the risk of allowing cement, which may be present in the channel 11 for fixation of the stem 94, to flow to the exterior side, creating there a risk of the cement flowing into and blocking the pores, thereby impeding ingrowth of bony material. In order to mitigate this risk, tongues 35 are provided. The tongues 35 are arranged at the adjacent side edges 21, 22 and/or sides of the leaf spring 30 and configured such as to cover the gaps formed by the compression slits 34. Between the tongues 35 and the leaf spring 30 a sealing gap 36 is provided which exists in any state of compression, i.e., in state of non-compression as well as in state of compression of the compressible joint 3, thereby forming a seal effective against unwanted passage of cement.

The sealing gap is preferably oriented such as to be parallel to the surface of the wall 2. The orientation of the gap is defined by the direction that a flow through the gap would take. Thereby, the width of the sealing gap is essentially unchanged by compression force, even if the compression slits 34 proper are shrinking considerably, and in conjunction with the compression slits 34 a kind of labyrinth seal is formed. This is shown in FIGS. 7A and 7B, wherein even in the compressed state (as shown in FIG. 7B) the sealing gap 36 maintains its width “S” as the compression slits 34 shrink to half “C/2” of their original width “C” under no or low compression force (as shown in FIG. 7A). Preferably, the tongues 35 are configured such as to form the sealing gap 36 being dimensioned such as to have a width of approximately 0.5 mm, preferably 0.4 mm or less. Thereby, an effective sealing against unwanted outflow of cement is achieved. As a result, the cement is kept inside the channel 11, and any unwanted influx into the pores provided on the outer face 27 of the wall 2 is effectively blocked.

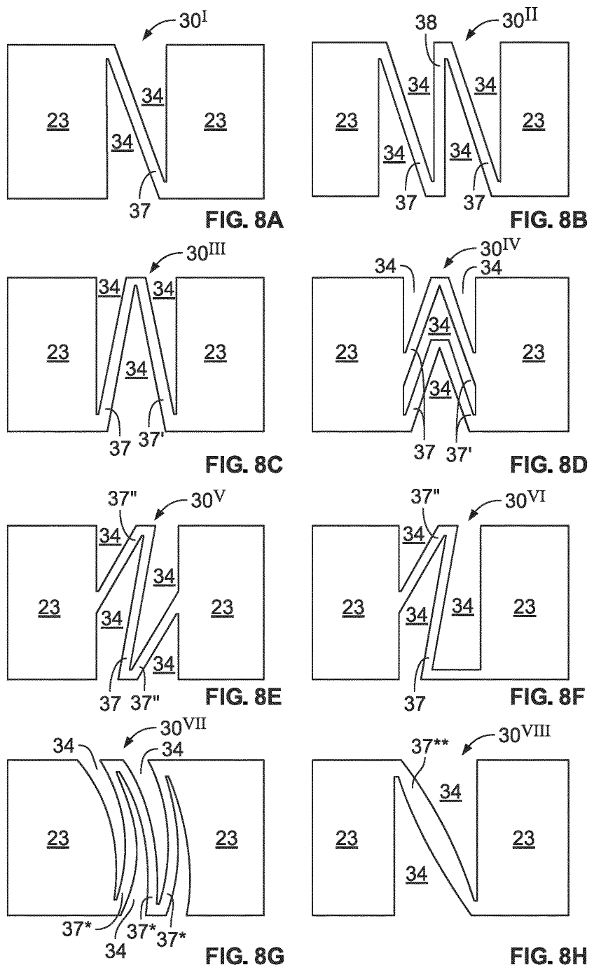

FIGS. 8A-H show various examples for different leaf springs 30 of the compressible joints 3. A rather simple example is shown in FIG. 8A, wherein the leaf spring 30I is basically configured as simple crossbar 37, as already discussed above. A corresponding view of the simple crossbar 37 example in a partly compressed state is shown in FIG. 6, already discussed above. FIG. 8B shows a second example for a leaf spring 30II having two of the crossbars 37 connected by an intermediate bar 38. This provides for a triplication of the effective length of the leaf spring, thereby making (ceteris paribus) the compressible joint 3 providing more travel and being softer. Similarly, FIG. 8C shows a third example for a leaf spring 30III featuring a second leg 37′ at the crossbar 37, embodied as a V- or folded Z-configuration. This provides for a doubling of the effective length of the leaf spring 3OIII, thereby making (ceteris paribus) this compressible joint to be softer and to provide more travel without needing the intermediate bar 38. In FIG. 8D a fourth example is shown having two of the folded Z-configurations of the third example shown in FIG. 8C, albeit each having a shorter effective length. Due to this shortening and the side-by-side configuration as shown, this leaf spring 3OIV provides more force opposing a compression and thus makes the compressible joint to become stiffer. In FIG. 8E a fifth example is shown which is similar to the third example, however one of the crossbars 37 is separated to two stubs, either stop being connected at one end to the remaining crossbar and the other end to an intermediate position at the adjacent side edge 21, 22, respectively. Combined length of the remaining crossbar and two stubs is approximatively similar to that of the two crossbars of the third example, and so is travel and softness of the resulting leaf spring 30V similar to that of the leaf spring 3OIII of the third example shown in FIG. 8C. In a sixth example as shown in FIG. 8F which is based on the fifth example as shown in FIG. 8E, one of the stubs 37″ remains and the other is rotated in plane to be oriented in the direction of compression, thereby becoming an essentially incompressible extension 39. As a result, the resulting leaf spring 3OVI is stiffer than that of the fifth example.

The elements like the crossbar(s) as used to form the leaf spring do not need to be straight elements (in uncompressed state). Various different configurations are possible, as already shown in the foregoing examples and further the elements may be differently shaped, e.g., be bent, rounded or curved. In FIG. 8G a seventh example for the leaf spring 3OVII having a plurality of curved elements forming a multiple folded configuration, in the depicted case a tri-fold configuration comprising three curved crossbars 37*. In terms of functionality, it is similar to the second example as shown in FIG. 8B. However, the curved crossbar 37* may provide for a better nesting in the compressed state, thereby improving handling of the medical implant. An eight example as shown in FIG. 8H is similar to the first example of FIG. 8A, however it features it features a leaf spring 3OVIII with a single crossbar 37** having two long sides which are rounded in a convex manner. Such a configuration allows i.e., for a smooth reduction of the width of the compression slits 34. Further, due to the increased width at the middle portion of the convex crossbar 37** such a configuration reduces the amount of free space between the side edges 21, 22. This reduces any risk of unwanted outflow of bone cement.

As already explained above, tongues 35 are to be provided in order to block bone cement from flowing out and eventually clogging the pores of the external porous structure 29. The tongues 35 are attached at the crossbar(s) forming the leaf spring 30 and/or the adjacent side edges 21, 22 of the respective neighboring wall segments 20. Size, configuration as well as attachment of the tongues 35 varies according to the constitution of the crossbars(s).

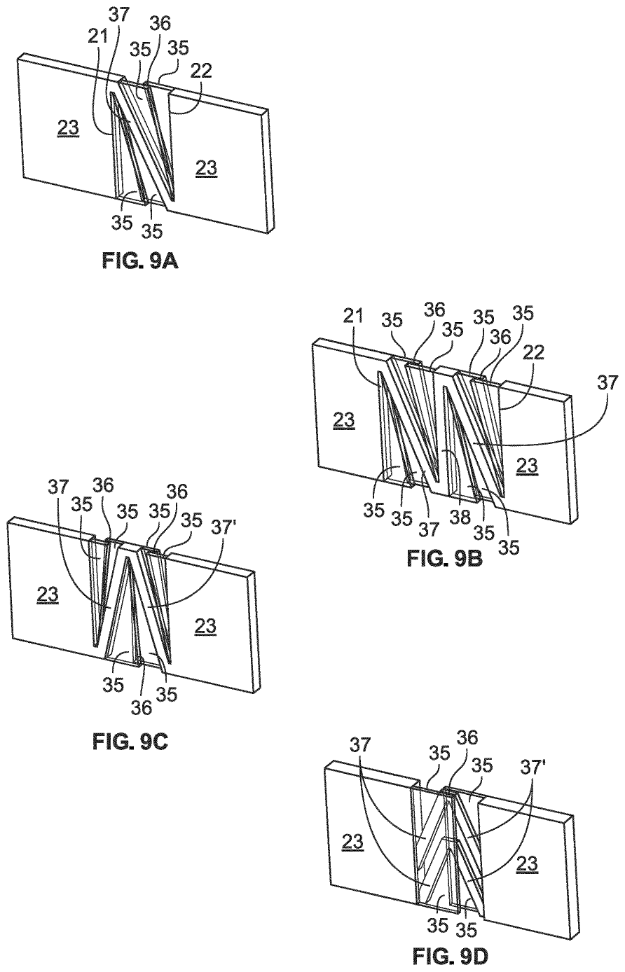

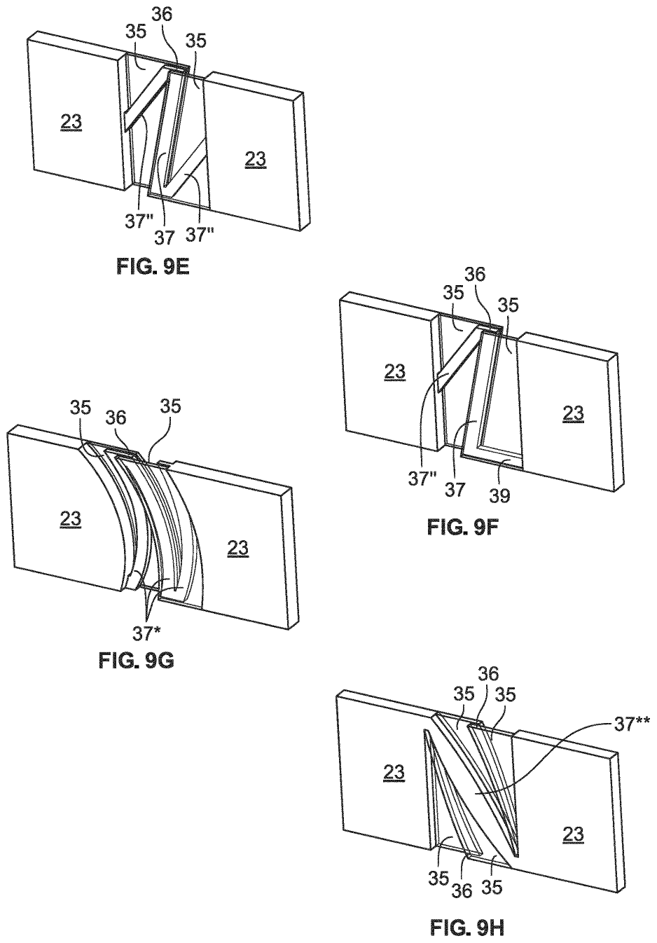

In FIGS. 9A to 9H perspective views for tongues to be provided for the examples as given in FIGS. 8A to 8H, respectively. The tongues 35 are shown in a semi-transparent manner for a better illustration of the relative arrangement between the tongues 35 and the respective crossbars 37.

FIG. 9A shows two pairs of tongues 35, each pair covering one of the compression slits 34 on either side of the leaf spring 30. The tongues of each pair are mounted at the side of the leaf spring 30, here crossbar 37, as well as at the respective side edge 21, 22 with a planar offset such that they can slide over each other. In the uncompressed state of the medical implant the tongues 35 overlap for a rather short distance in order to block any direct line-of-sight, which is sufficient to form the sealing gap 36 between said tongues 35 of each pair. Upon compression the side edges 21, 22 are moved toward each other, thereby shortening the distance to the crossbar. As a result, the tongues 35 of each pair are moved to a greater overlap, thereby ensuring proper sealing regardless of whether the medical implant is compressed or not. In the example of FIG. 9B the arrangement of the tongues is similar, the only difference is that two sets of tongue pairs are provided, one for each crossbar 37, and the respective inner tongues 35 of each pair being affixed to the intermediate bar 38. In the example of FIG. 9C three sets of tongue pairs are provided, the third pair being arranged between the two crossbars 37, 37′. An alternative example is depicted in FIG. 9D showing just one pair of tongues, each tongue 35 being attached to one of the side edges 21, 22, respectively. The example depicted in FIG. 9E is similar, the difference is mainly in the shape of the front free edge of either tongue. It is slanted in the same manner as the crossbar 37 is. The example of FIG. 9F follows basically the same principle, thereby also covering the crossbar 37, the stub 37″ and the extension 39 by just a single pair of tongues 35. This principle employing one pair of tongues 35 only can also be used for the example as depicted in FIG. 9G showing the curved crossbars 37* of FIG. 8G. For the convexly rounded crossbar 37**, on the other hand, in FIG. 9H an example is shown providing two pairs of tongues 35, similar to the example of FIG. 9A to which reference is made for brevity.

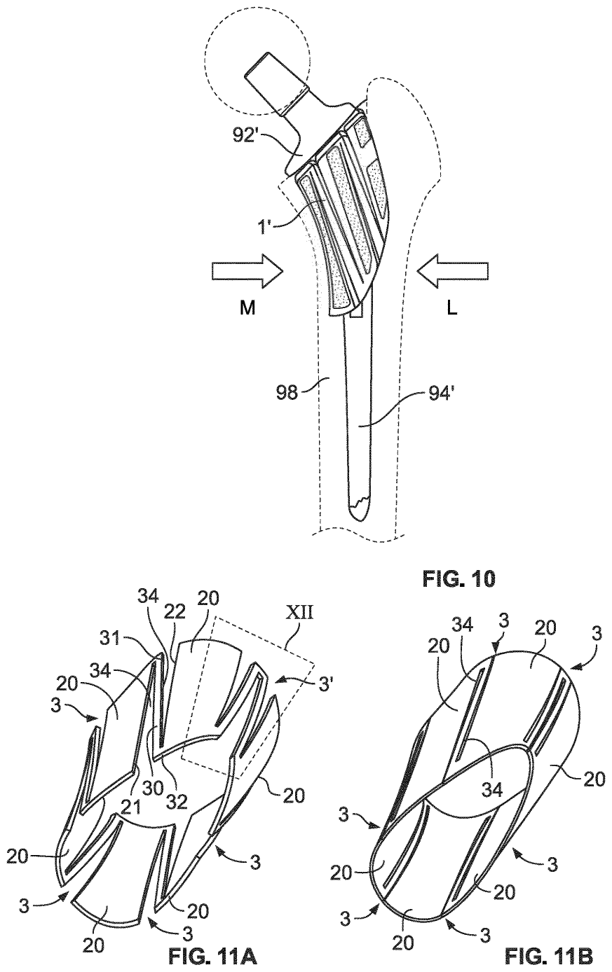

A second exemplary embodiment of the medical implant is shown in FIGS. 10-16. It is a sleeve configured to be applied to a proximal portion of a femur 98 in the context of a hip endoprostheses. Said sleeve may be placed around a stem 94′ of a femoral component 92′ of the hip endoprosthesis, as depicted in FIG. 10 showing a frontal view. Utilizing the common denomination scheme as used in anatomy, FIG. 10 shows a view of the left femur 98 from anterior in a direction toward the posterior. In FIG. 10, the letter “M” designates the medial side and the adjacent arrow marks a “medial view”, and the letter “L” designates the lateral side and the adjacent arrow marks a “lateral view” in standard anatomical terms as known to a person skilled in the art.

A perspective schematical view of a core of the wall 2 is shown in FIGS. 11A and 11B, wherein FIG. 11A shows the compressible joints 3 being in a non-compressed state and FIG. 11B shows the compressible joints 3 being in a compressed state. The configuration of the spring leaf 30 with its crossbars 37 having compression slits 34 on either side can be easily seen in FIG. 11A, whereas in the compressed state as shown in FIG. 11B the crossbars 37 are bended in plane such as to become nearly parallel to the adjacent side edges 21, 22, resulting in the compression slits 34 becoming very narrow in the fully compressed state.

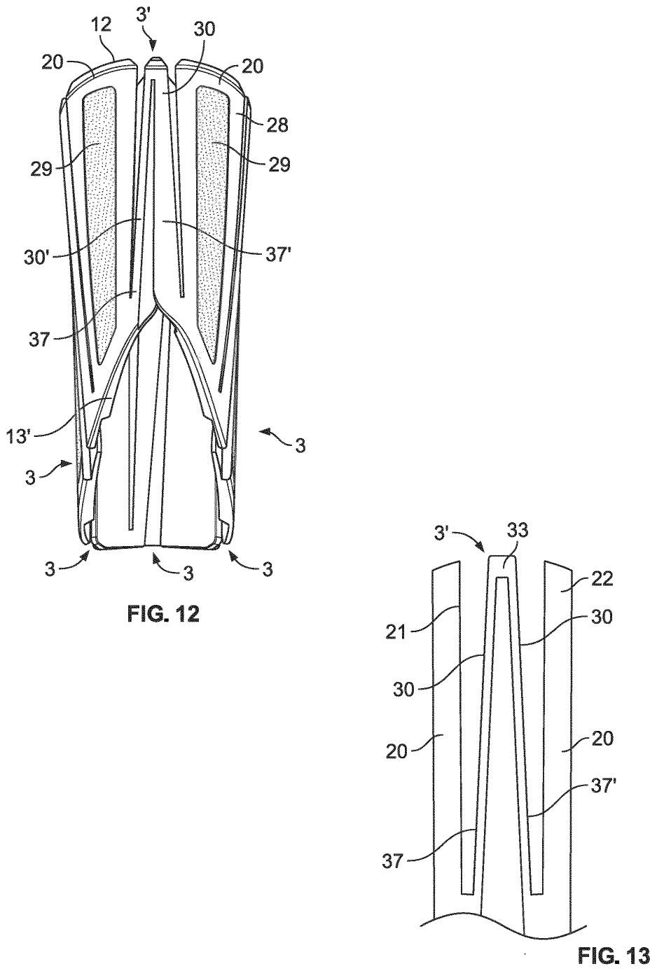

A lateral side view of the second exemplary embodiment is shown in FIG. 12. It differs from the first exemplary embodiment and its variant shown in FIGS. 1-6 mainly by the lower edge of the bottom of the sleeve being non-parallel to the upper edge of the top 12 of the sleeve, as it is the case with the first exemplary embodiment. Rather, according to this second exemplary embodiment, the lower edge 13′ of the sleeve is skewed to get closer to the upper edge at one side, the lateral side, of the medical implant.

Effectively, this results in the local height of the medical implant at this lateral side of the medical implant being reduced, whereas the local height is non-reduced at the medial side of medical implant which is opposite to the lateral side. The anterior and posterior (long) sides are tapering in height, as is can be seen in FIG. 10. Such a configuration having a reduced height at the lateral side is beneficial for a medical implant configured for implantation at a proximal end portion of the femur bone 98, such as to provide augmentation for a stem 94′ of the femoral part of a hip prosthesis.

Another important difference is shown in FIG. 13 showing a lateral detail view of a compressible joint 3′ of this second exemplary embodiment. Said compressible joint 3′ is positioned at the (short) lateral side and features a spring leaf 30′ having a second leg 37′ of the crossbar 37 thereby forming a V-or folded Z-configuration, as indicated in the example depicted in FIG. 8C. Thereby, as explained above with respect to said example of FIG. 8C, this provides for a doubled effective length of the leaf-spring of this compressible joint 3′, thereby making this compressible joint 3′ softer and increasing its travel. Additional crossbars as shown in FIG. 8B or FIG. 8G may be provided in a likewise manner in order to make the compressible joint even more soft and to further increase its travel, if so wished.

A particular benefit of this combination is that the softening effect of the special compressible joint 3′ having the doubled folded crossbar 37 counteracts the stiffening as it is a consequence of the reduced local height at said lateral side leading to crossbar(s) becoming shorter (since shortening of a spring makes it stiffer). In order to compensate this unwanted effect of such shortness-induced stiffening, the double crossbar forming the folded Z-shape crossbar 37 is provided, thereby achieving a softening effect. As a result, the wall 2 is to be compressed in a like manner at the lateral as well as the opposing medial side (having full, not reduced local height) which is a benefit for handling by the surgeon and further leads to improved distribution of force to the surrounding bone material, even for a complex shaped medical implant having varying local height.

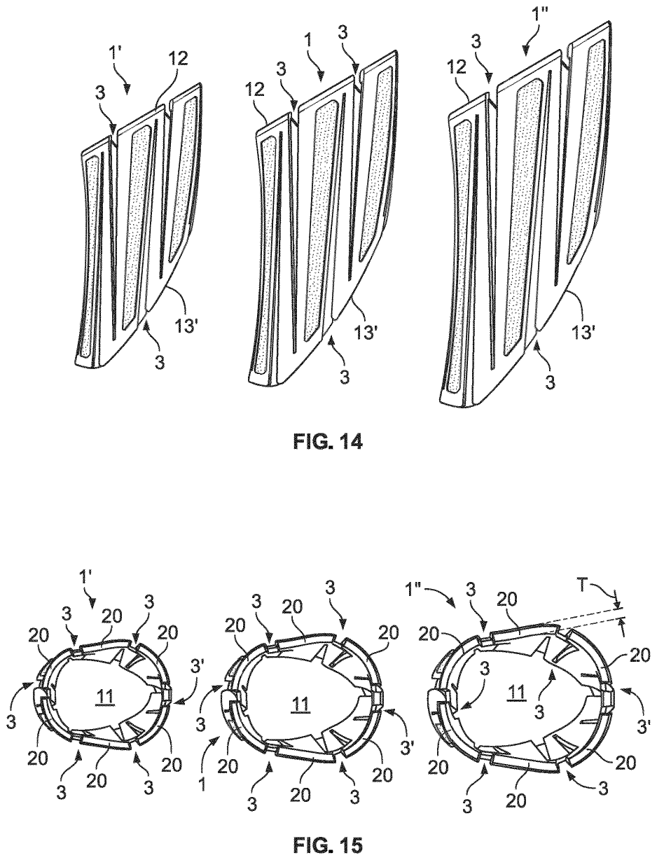

The medical implant may be provided in different sizes. A set of medical implants comprising a plurality of differently sized medical implants is shown in FIG. 14 to FIG. 16. In the depicted example, three different sizes are shown, however the number of different sizes is not limited to this number and can be two or four or more. Thereby, medical implants with proper dimensions can be provided for patients with different body constitutions. Typically, for a larger size the diameter/width of the medical implant is to be increased in contrast to a smaller size having a smaller diameter/width, as depicted in FIG. 15 which shows a top view of differently sized medical implants of the set as depicted in FIG. 14, namely a small sized implant 1′, a medium sized implant 1 and a large sized implant 1″. As it can be readily appreciated, for a larger size the height of the medical implant may be increased, too, as also depicted in FIG. 14.

Further, by providing medical implants with different shape,-e.g., greater or lower height and/or width, skewed upper or lower edges-different scenarios of bone defects can be addressed. Further, different sets having differently shaped medical implants can be provided for usage at different bones, e.g., medical implants configured to be used at the tibia bone 99, e.g., at a proximal portion of the tibia bone, or at the femur bone 98, e.g., at a proximal portion or the femur bone or, with yet another different shape, at the distal portion of the femur bone.

Concerning different sizes, there is however an issue with compression force or stiff-ness of the medical implants. When wall segments 20 manufactured employing similar material and having similar thickness “T” (see FIG. 15), a smaller sized implant 1′ tends to become stiffer than the larger sized implant 1″, i.e., requiring a higher compression force for achieving the same amount of compression travel.

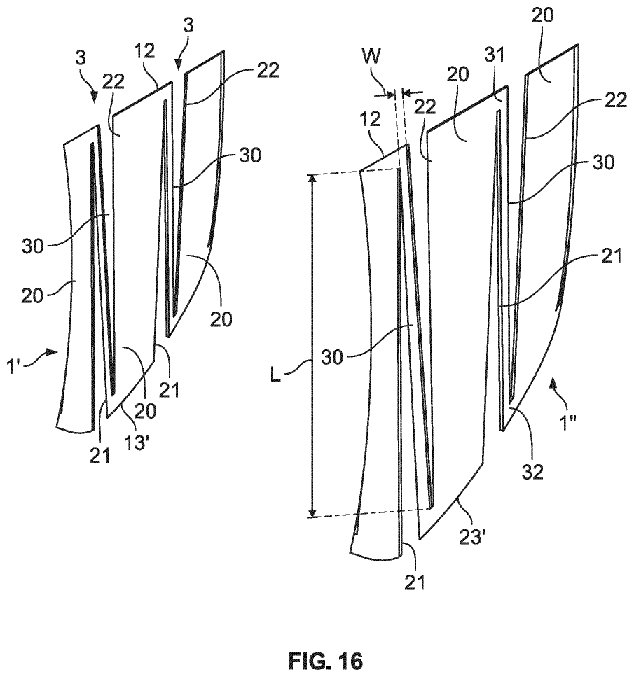

As it can be seen in FIG. 14 and particularly in FIG. 16, in a typical compressible joint 3 having its leaf spring 30 formed by one or more crossbars 37, the length L as well as the width W of the leaf spring of can be varied, as shown in FIG. 16 with respect to the crossbars 37 as an example. Preferably, within the set the smaller sized implant 1′ (see left portion of FIG. 16) features a crossbar 37 having a higher length/width ratio (L/W ratio) than that of the crossbar of the larger implant 1 (see right portion of FIG. 16). The higher length/width ratio may be achieved by a greater length or by- and this is often more practical-a reduced width of the leaf spring, or both. Thereby, the length/width ratio can be adjusted such that the differently sized implants 1, 1′, 1″ of the set require the same compression force. The differently sized implants then demonstrate the same elasticity to the surgeon which is a significant benefit. Likewise, it is thereby also possible to adjust the length/width ratio such that the smaller sized implant 1′ can be compressed more easily if so wished.

1. Medical implant, in particular for a joint endoprosthesis, being sleeve-shaped comprising a wall surrounding a channel extending through the sleeve (from a bottom to a top of the sleeve,
wherein
the wall is comprised of a plurality of wall segments arranged in a circumferential direction, wherein neighboring wall segments are connected at their adjacent edges facing each other by a compressible joint, the compressible joint being configured for reducing a separation distance between said neighboring wall segments of the wall and comprising a leaf spring spanning an interspace between the respective neighboring wall segments such as to form an elastic spring connection between the neighboring wall segments, the leaf spring being configured to provide opposing force against reduction of the separation distance.

2. The medical implant of claim 1, wherein said leaf spring is connected to at least one of the respective neighboring wall segments, at least one end of the leaf spring.

3. The medical implant of claim 1, wherein the leaf spring is positioned in the interspace between the respective adjacent edges.

4. The medical implant of claim 1, wherein at either side of the leaf spring a free space is provided forming compression slits, said compression slits being open at one side.

5. The medical implant of claim 4, wherein the compression slits are oriented in opposition to each other, wherein the compression slits comprise a V-shape.

6. The medical implant of claim 4, wherein the compression slits are covered by tongues provided at the adjacent edges and/or at the leaf spring.

7. The medical implant of claim 6, wherein a sealing gap is defined by the tongues having a width of 0.5 mm or less, or less, in an uncompressed state of the medical implant.

8. The medical implant of claim 1, wherein the leaf spring is formed as a unitary piece with at least one of the adjacent edges of the neighboring wall segments.

9. The medical implant of claim 1, wherein the leaf spring is configured as a crossbar.

10. The medical implant of claim 1, wherein two crossbars levers (37, 37′) of the leaf spring are connected at one of their respective ends and are connected at their other respective ends to the adjacent edges.

11. The medical implant of claim 1, wherein a plurality of said compressible joints are provided.

12. The medical implant of claim 11, wherein the compressible joints are located equidistantly and/or equiangularly along the circumference of the wall, and/or are located symmetrically to a middle plane of the medical implant.

13. The medical implant of any claim 1, wherein the wall has an inner face to the channel and an outer face on the opposite side of the wall, the outer face being at least partially comprised of porous material configured for bone ingrowth, wherein the inner face is solid.

14. Augment implant of claim 13, wherein pockets are provided comprising in which said porous material is affixed, the porous material being formed as a unitary piece with the wall.

15. The medical implant of claim 1, wherein the medical implant is made of biocompatible material, selected from a group consisting od zirconium, pure titanium, titanium grade 2, titanium grade 4, a titanium alloy, tantalum, cobalt chromium, and stainless steel.

16. The medical implant of claim 1, wherein the medical implant is formed by additive manufacturing.

17. The medical implant of claim 1, being configured as an augment device, wherein the channel is configured for receiving a stem of the endoprosthesis.

18. A set of medical implants of claim 1, comprising a plurality of said medical implants comprising at least a smaller medical implant (1′) and a larger medical implant (1″).

19. The set of medical implants according to claim 18, wherein the leaf spring of the smaller medical implant has a length/width ratio being higher than that of the leaf spring of the larger implant.

20. A method for treating a subject in need of bone repair, the method comprising implanting into the subject the medical implant of claim 1.
